(19) United States
(12) Patent Application Publication
(10) Pub. No.: US 2011/0311576 A1
(43) Pub. Date: Dec. 22, 2011

(54) MIXING LYOPHILISED MENINGOCOCCAL VACCINES WITH NON-HIB VACCINES

(75) Inventor: Mario Contorni, Siena (IT)

(73) Assignee: NOVARTIS AG, Basel (CH)

(21) Appl. No.: 13/139,043

(22) PCT Filed: Dec. 11, 2009
(86) PCT No.: PCT/IB09/07928

§ 371 Date: Sep. 7, 2011

(30) Foreign Application Priority Data
Dec. 11, 2008 (GB) 0822634.2

(51) Int. Cl. A61K 39/095; A61K 39/295; A61P 31/20; A61P 31/04; A61P 31/14; A61K 39/116; A61P 37/04

(52) U.S. Cl. 424/197.11

(57) ABSTRACT

An aqueous immunogen formulation is used to reconstitute a lyophilised component including conjugates of capsular saccharides from Neisseria meningitidis serogroups A, C, W135 and Y, thereby producing a combined vaccine. The aqueous formulation can include various immunogens but does not include a Hib conjugate.

This patent application claims priority from United Kingdom patent application 0822634.2, filed 11 Dec. 2008, the complete contents of which are incorporated herein by reference.

### TECHNICAL FIELD

This invention is in the field of formulating Neisseria meningitidis saccharide conjugates.

### BACKGROUND ART

Vaccines containing antigens from more than one pathogenic organism within a single dose are known as “multivalent” or “combination” vaccines e.g. diphtheria, tetanus & pertussis (“DTP”) vaccines and measles, mumps & rubella (“MMR”) vaccines. Combination vaccines offer patients the advantage of receiving a reduced number of injections, which leads to the clinical advantage of increased compliance (e.g. see chapter 29 of reference 1), particularly for pediatric vaccination. At the same time, however, they present difficulties due to factors including: physical and biochemical incompatibility between antigens and other components; immunological interference; and stability.

Some of these difficulties can be addressed by suitable formulation of the vaccine. For instance, the conjugated PRP capsular saccharide of Haemophilus influenzae type B (“Hib”) can be unstable in aqueous conditions and so Hib-containing vaccines in the INFANRIX™ series (including PEDIARIX™) include a lyophilised (freeze-dried) Hib component that is re-constituted at the time of use by an aqueous formulation of the remaining antigens. Reference 2 also describes the formulation of Hib-containing vaccines, and the Hib conjugate is lyophilised in combination with meningococcal conjugates, for extemporaneous reconstitution. In contrast, reference 3 describes fully-liquid formulations of meningococcal conjugates, in which further components (e.g. Hib or pneumococcal conjugates) may be lyophilised and reconstituted. Reference 91 describes combinations of meningococcal conjugates in which serogroup A (“MenA”) conjugates are lyophilised for reconstitution by liquid formulations of conjugates from other serogroups.

It is an object of the invention to provide further and improved formulations for vaccines including (a) capsular saccharide conjugates from multiple meningococcal serogroups and (b) at least one immunogen that is not a meningococcal capsular saccharide conjugate.

### DISCLOSURE OF THE INVENTION

According to the invention, an aqueous immunogen formulation is used to reconstitute a lyophilised component including conjugates of capsular saccharides from at least four different Neisseria meningitidis serogroups, thereby producing a combined vaccine. The aqueous formulation can include various immunogens but (unlike reference 4) does not include a Hib conjugate.

Thus the invention provides a kit comprising: (i) an aqueous component, comprising an immunogen, but not including a conjugate of a Haemophilus influenzae type B capsular saccharide; and (ii) a lyophilised component, comprising conjugates of capsular saccharides from meningococcal serogroups A, C, W135 and Y. For administration to a patient the aqueous and lyophilised components are combined to give a vaccine composition that is suitable for injection.

The invention also provides a method for preparing a combined vaccine, comprising the step of combining (i) an aqueous component, comprising an immunogen, but not including a conjugate of a Haemophilus influenzae type B capsular saccharide; and (ii) a lyophilised component, comprising conjugates of capsular saccharides from meningococcal serogroups A, C, W135 and Y.

The invention also provides a combined vaccine comprising: (i) conjugates of capsular saccharides from meningococcal serogroups A, C, W135 and Y; (ii) at least one further immunogen; and (iii) at least one lyophilisation stabiliser, provided that the vaccine does not include a conjugate of a Haemophilus influenzae type B capsular saccharide,

### The Aqueous Component

Kits and methods of the invention involve the use of an aqueous component that includes an immunogen other than a conjugate of a Hib saccharide. The aqueous component may, for example, include one of more of the following: (i) a diphtheria toxoid; (ii) a tetanus toxoid; (iii) a cellular B. pertussis antigen; (iv) at least one acellular B. pertussis antigen; (v) a hepatitis B virus antigen; (vi) an inactivated poliovirus vaccine (IPV); (vii) conjugated capsular saccharide(s) from at least one serotype of Streptococcus pneumoniae; (viii) vesicles from a serogroup B meningococcus; (ix) a hepatitis A virus (HAV) antigen; and/or (x) a human papillomavirus antigen.

The aqueous component does not include a capsular saccharide from Haemophilus influenzae type B. Usually it will also not include any meningococcal capsular saccharide(s). If it does include a meningococcal capsular saccharide, however, that saccharide will typically not be present in the lyophilised component.

### Diphtheria Toxoid

In a first embodiment, the aqueous component comprises a diphtheria toxoid.

Corynebacterium diphtheriae causes diphtheria. Diphtheria toxin can be treated (e.g. using formalin or formaldehyde) to remove toxicity while retaining the ability to induce specific anti-toxin antibodies after injection. These diphtheria toxoids are used in diphtheria vaccines, and are disclosed in more detail in chapter 13 of reference 1. Preferred diphtheria toxoids are those prepared by formaldehyde treatment. The diphtheria toxoid can be obtained by growing C. diphtheriae in growth medium (e.g. Fenton medium, or Linggoud & Fenton medium), which may be supplemented with bovine extract, followed by formaldehyde treatment, ultrafiltration and precipitation. The toxoided material may then be treated by a process comprising sterile filtration and/or dialysis.

Quantities of diphtheria toxoid can be expressed in international units (IU). For example, the NIBSC supplies the ‘Diphtheria Toxoid Adsorbed Third International Standard 1999’ [5,6], which contains 160 IU per ampoule. As an alternative to the IU system, the ‘Lf’ unit (“flocculating units” or the “limes flocculating dose”) is defined as the amount of toxoid which, when mixed with one International Unit of antitoxin, produces an optimally flocculating mixture [7]. For example, the NIBSC supplies ‘Diphtheria Toxoid, Plain’ [8], which contains 300 LF per ampoule, and also supplies ‘The 1st International Reference Reagent For Diphtheria Toxoid For Flocculation Test’ [9] which contains 900 LF per ampoule.

The aqueous component may include between 20 and 80 Lf/ml diphtheria toxoid, typically about 50 Lf/ml. By IU measurements, aqueous components will typically include at least 60 IU/ml. In other embodiments, however, lower concentrations may be used.

The diphtheria toxoid is usefully adsorbed onto an aluminium salt adjuvant, such as an aluminium hydroxide adjuvant or aluminium phosphate adjuvant.

### Tetanus Toxoid

In a second embodiment, the aqueous component comprises a tetanus toxoid.

Clostridium tetani causes tetanus. Tetanus toxin can be treated to give a protective toxoid. The toxoids are used in tetanus vaccines, and are disclosed in more detail in chapter 27 of reference 1. Preferred tetanus toxoids are those prepared by formaldehyde treatment. The tetanus toxoid can be obtained by growing C. tetani in growth medium (e.g. a Latham medium derived from bovine casein), followed by formaldehyde treatment, ultrafiltration and precipitation. The material may then be treated by a process comprising sterile filtration and/or dialysis.

Quantities of tetanus toxoid can be expressed in international units (IU). For example, the NIBSC supplies the ‘Tetanus Toxoid Adsorbed Third International Standard 2000’ [10,11], which contains 469 IU per ampoule. As an alternative to the IU system, the ‘Lf’ unit (“flocculating units” or the “limes flocculating dose”) is defined as the amount of toxoid which, when mixed with one International Unit of antitoxin, produces an optimally flocculating mixture [7]. For example, the NIBSC supplies ‘The 1st International Reference Reagent for Tetanus Toxoid For Flocculation Test’

which contains 1000 LF per ampoule.

The aqueous component may include between 5 and 50 Lf/ml tetanus toxoid, typically about 20 Lf/ml. By IU measurements, aqueous components will typically include at least 40 IU/ml. In other embodiments, however, lower concentrations may be used.

The tetanus toxoid is usefully adsorbed onto an aluminium salt adjuvant, such as an aluminium hydroxide adjuvant or aluminium phosphate adjuvant. This is not necessary, however, and adsorption of between 0-10% of the total tetanus toxoid can also be used.

### Pertussis Antigens

In a third embodiment, the aqueous component comprises a cellular B. pertussis antigen. In a fourth embodiment, the aqueous component comprises at least one acellular B. pertussis antigen. Bordetella pertussis causes whooping cough. Pertussis antigens in vaccines are either cellular (whole cell, in the form of inactivated B. pertussis cells) or acellular. Preparation of cellular pertussis antigens is well documented [e.g. see chapter 21 of ref.1] e.g. it may be obtained by heat inactivation of phase I culture of B. pertussis. As an alternative, however, acellular antigens can be used.

Where acellular antigen is used, this will usually include one, two or (preferably) three of the following purified antigens: (1) inactivated pertussis toxin (pertussis toxoid, or ‘PT’); (2) filamentous hemagglutinin (‘FHA’); (3) pertactin (also known as the ‘69 kiloDalton outer membrane protein’ or ‘PRN’). These three antigens can be prepared by isolation from B. pertussis culture grown in modified Stainer-Scholte liquid medium. Pertussis toxin and FHA can be isolated from the fermentation broth (e.g. by adsorption on hydroxyapatite gel), whereas pertactin can be extracted from the cells by heat treatment and flocculation (e.g. using barium chloride). The antigens can be purified in successive chromatographic and/or precipitation steps. Pertussis toxin and FHA can be purified by, for example, hydrophobic chromatography, affinity chromatography and size exclusion chromatography. Pertactin can be purified by, for example, ion exchange chromatography, hydrophobic chromatography and size exclusion chromatography. FHA and pertactin may be treated with formaldehyde prior to use according to the invention. Pertussis toxin may be inactivated (detoxified), to give PT, by treatment with formaldehyde and/or glutaraldehyde; as an alternative to this chemical detoxification procedure the PT may be a mutant toxin in which enzymatic activity has been reduced by mutagenesis [13] (e.g. the 9K/129G mutant [14]), but detoxification by chemical treatment is more usual.

These three pertussis antigens can be combined in any suitable ratio. The mass ratio of PT:FHA:p69 may, for example, be 1:1:1, 2:1:1, 3:4:4, 25:25:8 (as in the INFANRIX™ products), 16:16:5 (as in the BOOSTRIX™ products), 10:5:3 (as in the DAPTACEL™ product), 5:10:6 (as in the ADACEL™ product), etc.

Acellular pertussis antigens may be adsorbed onto one or more aluminium salt adjuvants. As an alternative, they may be added in an unadsorbed state. Where pertactin is added then it is preferably already adsorbed onto an aluminum hydroxide adjuvant. PT and FHA may be adsorbed onto an aluminum hydroxide adjuvant or an aluminum phosphate. Adsorption of all of PT, FHA and pertactin to aluminum hydroxide is useful.

The aqueous component may include: 1-100 μg/ml PT; 1-100 μg/ml FHA; and 1-50 μg/ml pertactin. For example, typical aqueous components may include (i) about 50 μg/ml PT, about 50 μg/ml FHA and about 16 μg/ml pertactin, or (ii) about 20 μg/ml PT, about 10 μg/ml FHA and about 6 μg/ml pertactin.

A useful aP mixture has 10 μg/ml PT (preferably 9K/129 G mutant), 5 μg/ml FHA and 5 μg/ml p69. Another useful aP mixture has 5 μg/ml PT (preferably 9K/129 G mutant), 2.5 μg/ml FHA and 2.5 μg/ml p69.

A useful adjuvanted aP mixture has 10 μg/ml PT (preferably 9K/129 G mutant), 5 μg/ml FHA, 5 μg/ml p69, 2 mg/ml aluminium hydroxide, 9 mg/ml sodium chloride and 0.1 mg/ml thimerosal. Another useful adjuvanted aP mixture has 5 μg/ml PT (preferably 9K/129 G mutant), 2.5 μg/ml FHA, 2.5 μg/ml p69, 2 mg/ml aluminium hydroxide, 9 mg/ml sodium chloride and 0.1 mg/ml thimerosal.

As well as PT, FHA and pertactin, it is possible to include fimbriae (e.g. agglutinogens 2 and 3) in an acellular pertussis vaccine. For example, about 10 μg/ml of fimbriae types 2 and 3 combined.

### Hepatitis B Virus Surface Antigen

In a fifth embodiment, the aqueous component comprises a hepatitis B virus (HBV) surface antigen (‘HBsAg’).

Hepatitis B virus (HBV) is one of the known agents that cause viral hepatitis. The HBV virion consists of an inner core surrounded by an outer protein coat or capsid, and the viral core contains the viral DNA genome. The major component of the capsid is a protein known as HBV surface antigen or, more commonly, ‘HBsAg’, which is typically a 226-amino acid polypeptide with a molecular weight of ˜24 kDa. All existing hepatitis B vaccines contain HBsAg, and when this antigen is administered to a normal vaccinee it stimulates the production of anti-HBsAg antibodies which protect against HBV infection.

For vaccine manufacture, FIBsAg has been made in two ways. The first method involves purifying the antigen in particulate form from the plasma of chronic hepatitis B carriers, as large quantities of HBsAg are synthesized in the liver and released into the blood stream during an HBV infection. The second way involves expressing the protein by recombinant DNA methods. HBsAg for use with the method of the invention is preferably recombinantly expressed in yeast cells. Suitable yeasts include, for example, Saccharomyces (such as S. cerevisiae) or Hanensula (such as H. polymorpha) hosts.

The HBsAg is usually non-glycosylated. Unlike native HBsAg (i.e. as in the plasma-purified product), yeast-expressed HBsAg is generally non-glycosylated, and this is the most preferred form of HBsAg for use with the invention, because it is highly immunogenic and can be prepared without the risk of blood product contamination.

The HBsAg will generally be in the form of substantially-spherical particles (average diameter of about 20 nm), including a lipid matrix comprising phospholipids. Yeast-expressed HBsAg particles may include phosphatidylinositol, which is not found in natural HBV virions. The particles may also include a non-toxic amount of LPS in order to stimulate the immune system [15]. HBsAg may be in the form of particles including a lipid matrix comprising phospholipids, phosphatidylinositol and polysorbate 20.

All known HBV subtypes contain the common determinant ‘a’. Combined with other determinants and subdeterminants, nine subtypes have been identified: ayw 1, ayw2, ayw3, ayw4, ayr, adw2, adw4, adrq− and adrq+. Besides these subtypes, other variants have emerged, such as HBV mutants that have been detected in immunised individuals (“escape mutants”). The usual HBV subtype with the invention is subtype adw2.

In addition to the ‘S’ sequence, a surface antigen may include all or part of a pre-S sequence, such as all or part of a pre-S1 and/or pre-S2 sequence.

Quantities of HBsAg are typically expressed in micrograms, and a typical concentration of HBsAg in the aqueous component is between 5 and 50 μg/ml e.g. about 20 μg/ml or about 40 μg/ml.

Although HBsAg may be adsorbed to an aluminium hydroxide adjuvant in the final vaccine (as in the well-known ENGERIX-B™ product), or may remain unadsorbed, it will generally be adsorbed to an aluminium phosphate adjuvant [16]. Other adjuvants may also be used, such as ‘ASO4’.

The aqueous component according to the fifth embodiment may be a commercially available product such as ENGERIX B™, RECOMBIVAX HB™ or FENDRIX™.

### Inactivated Poliovirus Vaccine

In a sixth embodiment, the aqueous component comprises an inactivated poliovirus vaccine (WV).

Poliovirus causes poliomyelitis. Rather than use oral poliovirus vaccine, the invention may use IPV, as disclosed in more detail in chapter 24 of reference 1.

Polioviruses may be grown in cell culture, and a preferred culture uses a Vero cell line, derived from monkey kidney. Vero cells can conveniently be cultured on microcarriers. After growth, virions may be purified using techniques such as ultrafiltration, diafiltration, and chromatography. Prior to administration to patients, polioviruses must be inactivated, and this can be achieved by treatment with formaldehyde.

Poliomyelitis can be caused by one of three types of poliovirus. The three types are similar and cause identical symptoms, but they are antigenically very different and infection by one type does not protect against infection by others. It is therefore preferred to use three poliovirus antigens in the invention: poliovirus Type 1 (e.g. Mahoney strain), poliovirus Type 2 (e.g. MEF-1 strain), and poliovirus Type 3 (e.g. Saukett strain). Sabin strains may also be used (e.g. see references 17 & 18). The viruses are preferably grown, purified and inactivated individually, and are then combined to give a bulk trivalent mixture for use with the invention.

Quantities of IPV are typically expressed in the ‘DU’ unit (the “D-antigen unit” [19]). It is usual to include between 1-100 DU per viral type in the aqueous component e.g. about 80 DU/ml of Type 1 poliovirus, about 16 DU/ml of type 2 poliovirus, and about 64 DU/ml of type 3 poliovirus. Lower doses can also be used, however, as disclosed in reference 20.

Poliovirus antigens are preferably not adsorbed to any aluminium salt adjuvant before being used with the invention, but they may become adsorbed onto aluminum adjuvant(s) during storage.

The aqueous component according to the sixth embodiment may be a commercially available product such as IPOL™

### Pneumococcal Saccharides

In a seventh embodiment, the aqueous component comprises conjugated capsular saccharide(s) from at least one serotype of Streptococcus pneumoniae.

The aqueous component will usually include capsular saccharide from more than one different pneumococcal serotypes. These are preferably prepared separately, conjugated separately, and then combined. Methods for purifying pneumococcal capsular saccharides are known in the art (e.g. see reference 21) and vaccines based on purified saccharides from 23 different serotypes have been known for many years. Improvements to these methods have also been described e.g. for serotype 3 as described in reference 22, or for serotypes 1, 4, 5, 6A, 6B, 7F and 19A as described in ref. 23.

Pneumococcal capsular saccharide(s) will typically be selected from the following serotypes: 1, 2, 3, 4, 5, 6A, 6B, 7F, 8, 9N, 9V, 10A, 11A, 12F, 14, 15B, 17F, 18C, 19A, 19F, 20, 22F, 23F and/or 33F. Thus, in total, an aqueous component may include a capsular saccharide from 1, 2, 3, 4, 5, 6, 7, 8, 9, 10, 11, 12, 13, 14, 15, 16, 17, 18, 19, 20, 21, 22, 23 or more different serotypes. Pneumococcal saccharide may be present at between 1 μg and 20 μg/ml (measured as saccharide) per serotype that is present.

A useful combination of serotypes is a 7-valent combination e.g. including capsular saccharide from each of serotypes 4, 6B, 9V, 14, 18C, 19F, and 23F. Another useful combination is a 9-valent combination e.g. including capsular saccharide from each of serotypes 1, 4, 5, 6B, 9V, 14, 18C, 19F and 23F. Another useful combination is a 10-valent combination e.g. including capsular saccharide from each of serotypes 1, 4, 5, 6B, 7F, 9V, 14, 18C, 19F and 23F. An 11-valent combination may further include saccharide from serotype 3. A 12-valent combination may add to the 10-valent mixture: serotypes 6A and 19A; 6A and 22F; 19A and 22F; 6A and 15B; 19A and 15B; or 22F and 15B. A 13-valent combination may add to the 11-valent mixture: serotypes 19A and 22F; 8 and 12F; 8 and 15B; 8 and 19A; 8 and 22F; 12F and 15B; 12F and 19A; 12F and 22F; 15B and 19A; 15B and 22F; 6A and 19A, etc.

Thus a useful 13-valent combination includes capsular saccharide from serotypes 1, 3, 4, 5, 6A, 6B, 7F, 9V, 14, 18C, 19, 19F and 23F e.g. prepared as disclosed in references 24, 25 and 26. One such combination includes serotype 6B saccharide at about 8μg/ml and the other 12 saccharides at concentrations of about 4μg/ml each. Another such combination includes serotype 6A and 6B saccharides at about 8 μg/ml each and the other 11 saccharides at about 4 μg/ml each.

Suitable carrier proteins for conjugates include bacterial toxins, such as diphtheria or tetanus toxins, or toxoids or mutants thereof. For example, the CRM197 diphtheria toxin mutant is useful [27]. Other suitable carrier proteins include synthetic peptides [28,29], heat shock proteins [30,31], pertussis proteins [32,33], cytokines [34], lymphokines [34], hormones [34], growth factors [34],artificial proteins comprising multiple human CD4⁺T cell epitopes from various pathogen-derived antigens [35] such as N19 [36], protein D from H. influenzae [37-39], pneumolysin [40] or its non-toxic derivatives [41], pneumococcal surface protein PspA [42], iron-uptake proteins [43], toxin A or B from C. difficile [44], recombinant Pseudomonas aeruginosa exoprotein A (rEPA) [45], the N. meningitidis OMPC outer membrane protein complex [46], etc.

Particularly useful carrier proteins for pneumococcal conjugate vaccines are CRM197, tetanus toxoid, diphtheria toxoid and H. influenzae protein D. CRM197 is used in PREVNAR™. A 13-valent mixture may use CRM197 as the carrier protein for each of the 13 conjugates, and CRM197 may be present at about 55-60 μg/ml.

Where the aqueous component includes conjugates from more than one pneumococcal serotype, it is possible to use the same carrier protein for each separate conjugate, or to use different carrier proteins. In both cases, though, a mixture of different conjugates will usually be formed by preparing each serotype conjugate separately, and then mixing them to form a mixture of separate conjugates. Reference 47 describes potential advantages when using different carrier proteins in multivalent pneumococcal conjugate vaccines, but the PREVNAR™ product successfully uses the same carrier for each of seven different serotypes.

A carrier protein may be covalently conjugated to a pneumococcal saccharide directly or via a linker. Various linkers are known. For example, attachment may be via a carbonyl, which may be formed by reaction of a free hydroxyl group of a modified saccharide with CDI [48,49] followed by reaction with a protein to form a carbamate linkage. Carbodiimide condensation can be used [50]. An adipic acid linker can be used, which may be formed by coupling a free —NH₂ group (e.g. introduced to a saccharide by amination) with adipic acid (using, for example, diimide activation), and then coupling a protein to the resulting saccharide-adipic acid intermediate [51,52].Other linkers include β-propionamido [53], nitrophenyl-ethylamine [54], haloacyl halides [55], glycosidic linkages [56], 6-aminocaproic acid [57], N-succinimidyl-3-(2-pyridyldithio)-propionate (SPDP) [58], adipic acid dihydrazide ADH [59], C₄ to C₁₂ moieties [60], etc.

Conjugation via reductive amination can be used. The saccharide may first be oxidised with periodate to introduce an aldehyde group, which can then form a direct covalent linkage to a carrier protein via reductive amination e.g. to the c-amino group of a lysine. If the saccharide includes multiple aldehyde groups per molecule then this linkage technique can lead to a cross-linked product, where multiple aldehydes react with multiple carrier amines. This cross-linking conjugation technique is particularly useful for at least pneumococcal serotypes 4, 6B, 9V, 14, 18C, 19F and 23F.

A pneumococcal saccharide may comprise a full-length intact saccharide as prepared from pneumococcus, and/or may comprise fragments of full-length saccharides i.e. the saccharides may be shorter than the native capsular saccharides seen in bacteria. The saccharides may thus be depolymerised, with depolymerisation occurring during or after saccharide purification but before conjugation. Depolymerisation reduces the chain length of the saccharides. Depolymerisation can be used in order to provide an optimum chain length for immunogenicity and/or to reduce chain length for physical manageability of the saccharides. Where more than one pneumococcal serotype is used then it is possible to use intact saccharides for each serotype, fragments for each serotype, or to use intact saccharides for some serotypes and fragments for other serotypes.

Where the aqueous component includes saccharide from any of serotypes 4, 6B, 9V, 14, 19F and 23F, these saccharides are preferably intact. In contrast, where the aqueous component includes saccharide from serotype 18C, this saccharide is preferably depolymerised.

A serotype 3 saccharide may also be depolymerised, For instance, a serotype 3 saccharide can be subjected to acid hydrolysis for depolymerisation [24] e.g. using acetic acid. The resulting fragments may then be oxidised for activation (e.g. periodate oxidation, maybe in the presence of bivalent cations e.g. with MgCl₂), conjugated to a carrier (e.g. CRM197) under reducing conditions (e.g. using sodium cyanoborohydride), and then (optionally) any unreacted aldehydes in the saccharide can be capped (e.g. using sodium borohydride) [24]. Conjugation may be performed in lyophilized material e.g. after co-lyophilizing activated saccharide and carrier.

A serotype 1 saccharide may be at least partially de-O-acetylated e.g. achieved by alkaline pH buffer treatment [25] such as by using a bicarbonate/carbonate buffer. Such (partially) de-O-acetylated saccharides can be oxidised for activation (e.g. periodate oxidation), conjugated to a carrier (e.g. CRM197) under reducing conditions (e.g. using sodium cyanoborohydride), and then (optionally) any unreacted aldehydes in the saccharide can be capped (e.g. using sodium borohydride) [25]. Conjugation may be performed in lyophilized material e.g. after co-lyophilizing activated saccharide and carrier.

A serotype 19A saccharide may be oxidised for activation (e.g. periodate oxidation), conjugated to a carrier (e.g. CRM197) in DMSO under reducing conditions, and then (optionally) any unreacted aldehydes in the saccharide can be capped (e.g. using sodium borohydride) [61]. Conjugation may be performed in lyophilized material e.g. after co-lyophilizing activated saccharide and carrier.

Pneumococcal conjugates can ideally elicit anticapsular antibodies that bind to the relevant saccharide e.g. elicit an anti-saccharide antibody level ≧0.20 μg/mL [62]. The antibodies may be evaluated by enzyme immunoassay (EIA) and/or measurement of opsonophagocytic activity (OPA). The EIA method has been extensively validated and there is a link between antibody concentration and vaccine efficacy.

The aqueous component according to the sixth embodiment may be a commercially available product such as PREVNAR™ or SYNFLORIX™ [63].

### Meningococcal Vesicles

In an eighth embodiment, the aqueous component comprises vesicles from a serogroup B meningococcus (‘MenB’).

Such vesicles include any proteoliposomic vesicle obtained by disrupting or blebbing from a meningococcal outer membrane to form vesicles therefrom that include protein components from the outer membrane. Thus the term includes OMVs (sometimes referred to as ‘blebs’), microvesicles (MVs [64]) and ‘native OMVs’ (‘NOMVs’ [65]).

MVs and NOMVs are naturally-occurring membrane vesicles that form spontaneously during bacterial growth and are released into culture medium. MVs can be obtained by culturing Neisseria in broth culture medium, separating whole cells from the smaller MVs in the broth culture medium (e.g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Strains for use in production of MVs can generally be selected on the basis of the amount of MVs produced in culture e.g. refs. 66 & 67 describe Neisseria with high MV production.

OMVs are prepared artificially from bacteria, and may be prepared using detergent treatment (e.g. with deoxycholate), or by non-detergent means (e.g. see reference 68). Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc., with sodium deoxycholate [69 & 70] being preferred for treating Neisseria) at a pH sufficiently high not to precipitate the detergent [71]. Other techniques may be performed substantially in the absence of detergent [68] using techniques such as sonication, homogenisation, microfluidisation, cavitation, osmotic shock, grinding, French press, blending, etc. Methods using no or low detergent can retain useful antigens such as NspA [68]. Thus a method may use an OMV extraction buffer with about 0.5% deoxycholate or lower e.g. about 0.2%, about 0.1%, <0.05% or zero.

A useful process for OMV preparation is described in reference 72 and involves ultrafiltration on crude OMVs, rather than instead of high speed centrifugation. The process may involve a step of ultracentrifugation after the ultrafiltration takes place.

Vesicles for use with the invention can be prepared from any serogroup B meningococcal strain. The strain may be of any serotype (e.g. 1, 2a, 2b, 4, 14, 15, 16, etc.), any serosubtype, and any immunotype (e.g. L1; L2; L3; L3,3,7; L10; etc.). The meningococci may be from any suitable lineage, including hyperinvasive and hypervirulent lineages e.g. any of the following seven hypervirulent lineages: subgroup I; subgroup III; subgroup IV-1; ET-5 complex; ET-37 complex; A4 cluster; lineage 3. These lineages have been defined by multilocus enzyme electrophoresis (MLEE), but multilocus sequence typing (MLST) has also been used to classify meningococci [ref. 73] e.g. the ET-37 complex is the ST-11 complex by MLST, the ET-5 complex is ST-32 (ET-5), lineage 3 is ST-41/44, etc. Vesicles can be prepared from strains having one of the following subtypes: P1.2; P1.2,5; P1.4; P1.5; P1.5,2; P1.5,c; P1.5c,10; P1.7,16; P1.7,16b; P1.7h,4; P1.9; P1.15; P1.9,15; P1.12,13; P1.13; P1.14; P1.21,16; P1.22,14.

Vesicles used with the invention may be prepared from wild-type meningococcal strains or from mutant meningococcal strains. For instance, reference 74 discloses preparations of vesicles obtained from N. meningitidis with a modified fur gene. Reference 80 teaches that nspA expression should be up-regulated with concomitant porA and cps knockout. Further knockout mutants of N. meningitidis for OMV production are disclosed in references 80 to 82. Reference 75 discloses vesicles in which fHBP is upregulated. Reference 76 discloses the construction of vesicles from strains modified to express six different PorA subtypes. These or others mutants can all be used with the invention.

Thus a strain used with the invention may in some embodiments express more than one PorA subtype. 6-valent and 9-valent PorA strains have previously been constructed. The strain may express 2, 3, 4, 5, 6, 7, 8 or 9 of PorA subtypes: P1.7,16; P1.5-1,2-2; P1.19,15-1; P1.5-2,10; P1.12-1,13; P1.7-2,4; P1.22,14; P1.7-1,1 and/or P1.18-1,3,6. In other embodiments, however, a strain may have been down-regulated for PorA expression e.g. in which the amount of PorA has been reduced by at least 20% (e.g. >30%, >40%, >50%, >60%, >70%, >80%, >90%, >95%, etc.), or even knocked out, relative to wild-type levels (e.g. relative to strain H44/76, as disclosed in ref. 80).

In some embodiments a strain may hyper-express (relative to the corresponding wild-type strain) certain proteins. For instance, strains may hyper-express NspA, protein 287 [77], fHBP [75], TbpA and/or TbpB [78], Cu,Zn-superoxide dismutase [78], etc.

In some embodiments a strain may include one or more of the knockout and/or hyper-expression mutations disclosed in references 79 to 82. Useful genes for down-regulation and/or knockout include: (a) Cps, CtrA, CtrB, CtrC, CtrD, FrpB, GalE, HtrB/MsbB, LbpA, LbpB, LpxK, Opa, Opc, PilC, PorB, SiaA, SiaB, SiaC, SiaD, TbpA, and/or TbpB [79]; (b) CtrA, CtrB, CtrC, CtrD, FrpB, GalE, HtrB/MsbB, LbpA, LbpB, LpxK, Opa, Opc, PhoP, Pi1C, PmrE, PmrF, SiaA, SiaB, SiaC, SiaD, TbpA, and/or TbpB [80]; (c) ExbB, ExbD, rmpM, CtrA, CtrB, CtrD, GalE, LbpA, LpbB, Opa, Opc, PilC, PorB, SiaA, SiaB, SiaC, SiaD, TbpA, and/or TbpB [81]; and (d) CtrA, CtrB, CtrD, FrpB, OpA, OpC, PilC, PorB, SiaD, SynA, SynB, and/or SynC [82].

Where a mutant strain is used, in some embodiments it may have one or more, or all, of the following characteristics: (i) up-regulated TbpA; (ii) up-regulated NhhA; (iii) up-regulated Omp85; (iv) up-regulated LbpA; (v) up-regulated NspA; (vi) knocked-out PorA; (vii) down-regulated or knocked-out FrpB; (viii) down-regulated or knocked-out Opa; (ix) down-regulated or knocked-out Opc; (x) deleted cps gene complex; (xi) down-regulated or knocked out LgtB; (xii) down-regulated or knocked-out LgtA and/or LgtC; (xiii) down-regulated or knocked-out LgtE; (xiv) down-regulated or knocked-out GalE. Knock-out is preferred to down-regulation.

Meningococcal lipooligosaccharide (LOS) in a vesicle can be treated so as to link the LOS and protein components in the vesicle (“intra-bleb” conjugation [82]).

LOS may be O-acetylated on a GlcNac residue attached to its Heptose II residue e.g. for L3 [83].

An aqueous component can include more than one type of LOS e.g. LOS from meningococcal immunotypes L2 and L3. For example, the LOS combinations disclosed in reference 84 may be used.

In some embodiments of the invention, the aqueous component is not one of the antigen mixtures which include meningococcal serogroup B proteins as disclosed in reference 3.

The aqueous component according to the eighth embodiment may be a commercially available product such as MENZB™, HEXAMEN™ or NONAMEN™.

### Hepatitis A Virus Antigens

In a ninth embodiment, the aqueous component comprises a hepatitis A virus (HAV) antigen.

HAV is one of the known agents which causes viral hepatitis. HAV vaccines are disclosed in chapter 15 of reference 1. A useful HAV component is based on inactivated virus, and inactivation can be achieved by formalin treatment. Virus can be grown on human embryonic lung diploid fibroblasts, such as MRC-5 cells. A useful HAV strain is HM175, although CR326F can also be used. The cells can be grown under conditions that permit viral growth. The cells are lysed, and the resulting suspension can be purified by ultrafiltration and gel permeation chromatography.

The amount of HAV antigen, measured in EU (Elisa Units), in an aqueous component is typically at least about 500 EU/ml.

The aqueous component according to the ninth embodiment may be a commercially available product such as HAVRIX™, AVAXIM™ or VAQTA™.

### Human Papillomavirus Antigen

In a tenth embodiment, the aqueous component comprises a human papillomavirus (HPV) antigen.

HPV is a cause of cervical cancer. A preferred HPV antigen for inclusion in the aqueous component is the HPV L1 capsid protein, which can assemble to form structures known as virus-like particles (VLPs). The VLPs can be produced by recombinant expression of L1 in yeast cells (e.g. in S. cerevisiae) or in insect cells (e.g. in Spodoptera cells, such as S. frugiperda, or in Drosophila cells). For yeast cells, plasmid vectors can carry the L1 gene(s); for insect cells, baculovirus vectors can carry the L1 gene(s). More preferably, the aqueous component includes L1 VLPs from both HPV-16 and HPV-18 strains. In addition to HPV-16 and HPV-18 strains, it is also possible to include L1 VLPs from HPV-6 and HPV-11 strains. The use of oncogenic HPV strains is also possible. A vaccine may include between 20-60 m/ml (e.g. about 40 μg/ml) of L1 per HPV strain.

The aqueous component according to the tenth embodiment may be a commercially available product such as GARDASIL™ or CERVARIX™

### Combinations

The aqueous component may comprise combinations of the immunogens listed for the first to tenth embodiments. Thus, for example, the aqueous component may comprise:
A diphtheria toxoid and a tetanus toxoid.
A diphtheria toxoid, a tetanus toxoid and a cellular B. pertussis antigen.
A diphtheria toxoid, a tetanus toxoid and an acellular B. pertussis antigen.
A diphtheria toxoid, a tetanus toxoid, a cellular B. pertussis antigen, and HBsAg.
A diphtheria toxoid, a tetanus toxoid, an acellular B. pertussis antigen, and HBsAg.
A diphtheria toxoid, a tetanus toxoid, a cellular B. pertussis antigen, and IPV.
A diphtheria toxoid, a tetanus toxoid, an acellular B. pertussis antigen, and IPV.
A diphtheria toxoid, a tetanus toxoid, a cellular B. pertussis antigen, HBsAg and IPV.
A diphtheria toxoid, a tetanus toxoid, an acellular B. pertussis antigen, HBsAg and IPV.
Vesicles from a serogroup B meningococcus and conjugated capsular saccharide(s) from at least one serotype of Streptococcus pneumoniae [85].
A HAV antigen and HBsAg.

Thus, for example, the aqueous component could be one of the following marketed products: INFANRIX™; DAPTACEL™; INFANRIX HIB™; INFANRIX HEPB™; INFANRIX PENTA™; PEDIARIX™; TWINRIX™; TRITANRIX™; QUINTANRIX™; IMOVAX POLIO™; TRIVAC HB™; TRIPEDIA™; TRITANRIX HEPB™; ECOVAC™; ZILBRIX™; etc.

A useful aqueous component may have 20 Lf/ml tetanus toxoid, 50 Lf/ml diphtheria toxoid, 10 μg/ml PT (preferably 9K/129 G mutant), 5 μg/ml FHA and 5 μg/ml p69. Another useful aqueous component may have 10 Lf/ml tetanus toxoid, 25 Lf/ml diphtheria toxoid, 5 μg/ml PT (preferably 9K/129 G mutant), 2.5 μg/ml FHA and 2.5 μg/ml p69. Another useful aqueous component may have 10 Lf/ml tetanus toxoid, 30 Lf/ml diphtheria toxoid, 5 μg/ml PT (preferably 9K/129 G mutant), 2.5 μg/ml FHA and 2.5 μg/ml p69. Another useful aqueous component may have 20 Lf/ml tetanus toxoid, 50 Lf/ml diphtheria toxoid, 5 μg/ml PT (preferably 9K/129 G mutant), 2.5 μg/ml FHA and 2.5 μg/ml p69. Another useful aqueous component may have 10 Lf/ml tetanus toxoid, 5 Lf/ml diphtheria toxoid, 10 μg/ml PT (preferably 9K/129 G mutant), 5 μg/ml FHA and 5 μg/ml p69. Another useful aqueous component may have 10 Lf/ml tetanus toxoid, 4 Lf/ml diphtheria toxoid, 5 μg/ml PT (preferably 9K/129 G mutant), 2.5 μg/ml FHA and 2.5 μg/ml p69. Another useful aqueous component may have between 5-15 Lf/ml tetanus toxoid, between 2-8 Lf/ml diphtheria toxoid, between 1-20 μg/ml PT preferably as the 9K/129 G mutant, between 1-20 μg/ml FHA, and 1-20 μg/ml p69.

### The Lyophilised (Freeze-Dried) Component

Kits and methods of the invention involve the use of a lyophilised component that includes conjugates of meningococcal capsular saccharides from at least serogroups A, C, W135 and Y.

Administration of the meningococcal conjugates preferably results in a bactericidal antibody response, with an increase in serum bactericidal assay (SBA) titre for the relevant serogroup of at least 4-fold, and preferably at least 8-fold, measured with human complement [86]. If rabbit complement is used to measure SBA titres then the titre increase is preferably at least 128-fold.

Conjugated monovalent vaccines against serogroup C have been approved for human use, and include MENJUGATE™ [87], MENINGITEC™ and NEISVAC-C™. Mixtures of conjugates from serogroups A+C are known [88,89] and mixtures of conjugates from serogroups A+C+W135+Y have been reported [90-93] and were approved in 2005 as the aqueous MENACTRA™ product. The lyophilised component used with the invention include saccharides from serogroups A, C, W135 and Y.

In some embodiments, the A, C, W135 and Y saccharides are present at substantially equal masses e.g. the mass of each serogroup's saccharide is within ±5% of each other. In other embodiments, however, the mass of saccharide from one serogroup may differ from the mass of saccharide in another serogroup e.g. one serogroup may have a dose 2× that of another serogroup. A typical quantity of saccharide per serogroup is between 1 μg and 20 μg e.g. between 2 and 10 μg. For an individual serogroup the mass of saccharide per vaccine dose (e.g. per final 0.5 ml volume) may be, for example, about 2.5 μg, about 4 μg, about 5 μg or about 10 μg. Examples of suitable A:C:W135:Y mass ratios are 1:1:1:1, 2:1:1:1, 1:4:1:1, 1:2:1:1 & 2:2:1:1.

The capsular saccharide of serogroup A meningococcus is a homopolymer of (α1→6)-linked N-acetyl-D-mannosamine-1 -phosphate, with partial O-acetylation in the C3 and C4 positions. Acetylation at the C-3 position can be 70-95%. Conditions used to purify the saccharide can result in de-O-acetylation (e.g. under basic conditions), but it is useful to retain OAc at this C-3 position. In some embodiments, at least 50% (e.g. at least 60%, 70%, 80%, 90%, 95% or more) of the mannosamine residues in a serogroup A saccharides are O-acetylated at the C-3 position. Acetyl groups can be replaced with blocking groups to prevent hydrolysis [94], and such modified saccharides are still serogroup A saccharides within the meaning of the invention.

The serogroup C capsular saccharide is a homopolymer of (α2→9)-linked sialic acid (N-acetyl neuraminic acid, or ‘NeuNAc’). The saccharide structure is written as →9)-Neu p NAc 7/8 OAc-(α2→. Most serogroup C strains have O-acetyl groups at C-7 and/or C-8 of the sialic acid residues, but about 15% of clinical isolates lack these. O-acetyl groups [95,96].The presence or absence of OAc groups generates unique epitopes, and the specificity of antibody binding to the saccharide may affect its bactericidal activity against O-acetylated (OAc−) and de-O-acetylated (OAc+) strains [97-99]. Serogroup C saccharides used with the invention may be prepared from either OAc+ or OAc− strains. Licensed MenC conjugate vaccines include both OAc− (NEISVAC-C™) and OAc+ (MENJUGATE™ & MENINGITEC™) saccharides. In some embodiments, strains for production of serogroup C conjugates are OAc+ strains, e.g. of serotype 16, serosubtype P1.7a, 1, etc. Thus C:16:P1.7a,1 OAc+ strains may be used. OAc+ strains in serosubtype P1.1 are also useful, such as the C11 strain.

The serogroup W135 saccharide is a polymer of sialic acid-galactose disaccharide units. Like the serogroup C saccharide, it has variable O-acetylation, but at sialic acid 7 and 9 positions [100]. The structure is written as: →4)-D-Neup5Ac(7/9OAc)-α-(2→6)-D-Gal-α-(1→. The serogroup W135 saccharides used according to the invention may have the same degree of O-acetylation as seen in native serogroup W135 capsular saccharides, or they may be partially or totally de-O-acetylated at one or more positions of the saccharide ring, or they may be hyper-O-acetylated relative to the native capsular saccharides. In some embodiments, no more than 50% (e.g. at most 40%, 30%, 20%, or 10%; for example, between 40% and 45%) of the sialic acid residues in a serogroup W135 saccharide are O-acetylated at the C-7 and/or C-9 position(s).

The serogroup Y saccharide is similar to the serogroup W135 saccharide, except that the disaccharide repeating unit includes glucose instead of galactose. Like serogroup W135, it has variable O-acetylation at sialic acid 7 and 9 positions [100]. The serogroup Y structure is written as: →4)-D-Neup5Ac(7/9OAc)-α-(2→6)-D-Glc-α-(1→. The serogroup Y saccharides used according to the invention may have the same degree of O-acetylation as seen in native serogroup Y capsular saccharides, or they may be partially or totally de-O-acetylated at one or more positions of the saccharide ring, or they may be hyper-O-acetylated relative to the native capsular saccharides. In some embodiments, no more than 50% (e.g. at most 40%, 30%, 20%, or 10%; for example, between 30% and 40%) of the sialic acid residues in a serogroup Y saccharide are O-acetylated at the C-7 and/or C-9 position(s).

The saccharide moieties in conjugates may comprise full-length saccharides as prepared from meningococci, and/or may comprise fragments of full-length saccharides i.e. the saccharides may be shorter than the native capsular saccharides seen in bacteria. The saccharides may thus be depolymerised, with depolymerisation occurring during or after saccharide purification but before conjugation. Depolymerisation reduces the chain length of the saccharides. One depolymerisation method involves the use of hydrogen peroxide [90]. Hydrogen peroxide is added to a saccharide (e.g. to give a final H₂O₂ concentration of 1%), and the mixture is then incubated (e.g. at about 55° C.) until a desired chain length reduction has been achieved. Another depolymerisation method involves acid hydrolysis [91]. Other depolymerisation methods are known in the art. The saccharides used to prepare conjugates for use according to the invention may be obtainable by any of these depolymerisation methods. Depolymerisation can be used in order to provide an optimum chain length for immunogenicity and/or to reduce chain length for physical manageability of the saccharides. In some embodiments, saccharides have the following range of average degrees of polymerisation (Dp): A=10-20; C=12-22; W135=15-25; Y=15-25. In terms of molecular weight, rather than Dp, useful ranges are, for all serogroups: <100 kDa; 5 kDa-75 kDa; 71 kDa-50 kDa; 8 kDa-35 kDa; 12 kDa-25 kDa; 15 kDa-22 kDa.

The saccharides used according to the invention may be O-acetylated with the same O-acetylation pattern as seen in native capsular saccharides, or they may be partially or totally de-O-acetylated at one or more positions of the saccharide rings, or they may be hyper-O-acetylated relative to the native capsular saccharides.

Useful carrier proteins (see below) include CRM197, diphtheria toxoid and/or tetanus toxoid. Where the lyophilised component includes conjugates from more than one meningococcal serogroup then the various conjugates may use different carrier proteins (e.g. one serogroup on CRM197, another on tetanus toxoid) or they may use the same carrier protein (e.g. saccharides from two serogroups separately conjugated to CRM197 and then combined).

Suitable meningococcal conjugates can be made by the methods disclosed in, for example, any of references 90, 91, 101, 102, 103, 104, 105, 106, 107, 150 and/or 151, or by any other suitable method.

A preferred lyophilised component includes the meningococcal conjugates from serogroups A, C, W135 and Y as described in references 105 and 108 (the full contents of both of which are incorporated by reference herein).

Another useful lyophilised component is unadjuvanted and includes 5 μg of capsular saccharide for each of serogroups A, C, W135 and Y, with each serogroup's saccharide being separately conjugated to a tetanus toxoid carrier, as described in reference 109 (the full contents of which are incorporated by reference herein.

As an alternative to purifying saccharides from bacteria, saccharides may be prepared by chemical synthesis, in full or in part [110,111].

For stability reasons, a lyophilised component may include a stabiliser such as lactose, sucrose, trehalose or mannitol, as well as mixtures thereof e.g. lactose/sucrose mixtures, sucrose/mannitol mixtures, etc. Using a sucrose/mannitol mixture can speed up the drying process.

A lyophilised component may also include sodium chloride.

Soluble components in the lyophilised material will be retained in the composition (combined vaccine) after reconstitution. Thus the final combined vaccine may contain one or more such stabilisers (e.g. may include lactose and/or sucrose) and may contain sodium chloride.

The lyophilised component may or may not include an adjuvant, such as an aluminium salt.

The lyophilised component will usually be free from one or more (and preferably all six) of: (i) Bordetella pertussis antigens; (ii) HBsAg; (iii) inactivated poliovirus; (iv) vesicles from a serogroup B meningococcus; (v) hepatitis A virus antigen; and/or (vi) a HPV antigen. It will also usually be free from diphtheria toxoid and tetanus toxoid, except for any toxoid(s) that have been used as carrier proteins during conjugation of the meningococcal conjugate(s). In some embodiments the lyophilised component includes a conjugated capsular saccharide from Haemophilus influenzae type B; in other embodiments the lyophilised component does not include a conjugated capsular saccharide from Haemophilus influenzae type B.

Seven specific embodiments of the lyophilised component include: (a) a mixture comprising saccharides from serogroups A, C, W135 and Y, each separately conjugated to CRM197, to give a final vaccine dose of 10 μg for serogroup A and 5 μg for serogroups C, W135 & Y; (b) a mixture comprising saccharides from serogroups A, C, W135 and Y, each separately conjugated to diphtheria toxoid, to give a final vaccine dose of 5 μg for each serogroup; (c) a mixture comprising saccharides from serogroups A, C, W135 and Y, each separately conjugated to tetanus toxoid, to give a final vaccine dose of 2.5 μg for each serogroup; (d) a mixture comprising saccharides from serogroups A, C, W135 and Y, each separately conjugated to tetanus toxoid, to give a final vaccine dose of 5 μg for each serogroup; (e) a mixture comprising saccharides from serogroups A, C, W135 and Y, each separately conjugated to tetanus toxoid, to give a final vaccine dose of 2.5 μg for serogroups A, W135 and Y and 10 μg for serogroup C; (f) a mixture comprising saccharides from serogroups A, C, W135 and Y, each separately conjugated to tetanus toxoid, to give a final vaccine dose of 2.5 μg for serogroups A, W135 and Y and 5 μg for serogroup C; and (g) a mixture comprising saccharides from serogroups A, C, W135 and Y, each separately conjugated to tetanus toxoid, to give a final vaccine dose of 2.5 μg for serogroups W135 and Y and 5 μg for serogroups A and C.

### Packaging Vaccines of the Invention

The wet and dry components used with the invention must be kept separate from each other prior to use. Thus they are packaged separately in the form of a kit. The kit can take various forms.

In some embodiments, the two components are packaged into separate containers. In other embodiments, the two components are packaged into separate chambers of a single container e.g. into separate containers of a multi-chamber syringe. A dual-chamber syringe allows two individual compositions to be kept separately during storage, but to be mixed as the syringe plunger is activated.

Lyophilised material will usually be presented in a sealed vial. The vial will have an opening (e.g. a rubber seal, a breakable neck, etc.) that can maintain sterility while permitting removal of its contents and/or introduction of aqueous material for reconstitution. Vials can be made of various materials e.g. of a glass, of a plastic, etc.

Aqueous material may also be presented in a vial, but as an alternative may be presented in e.g. a syringe. Again, the container will be able to maintain sterility while permitting removal of its contents. A syringe may be applied with or without a needle attached to it; in the latter case, a separate needle may be packaged with the syringe for assembly and use, and the syringe will generally have a tip cap to seal the tip prior to attachment of a needle. Safety needles are preferred. 1-inch 23-gauge, 1-inch 25-gauge and 5/8-inch 25-gauge needles are typical. The plunger in a syringe may have a stopper to prevent the plunger from being accidentally removed during aspiration. Syringes can be made of various materials e.g. of a glass, of a plastic, etc.

A vial can have a cap (e.g. a Luer lock) adapted such that a syringe can be inserted into the cap, the contents of the syringe can be expelled into the vial (to reconstitute lyophilised material therein), and the contents of the vial can be removed back into the syringe. After removal of the syringe from the vial, a needle can then be attached and the composition can be administered to a patient. The cap may be located inside a seal or cover, such that the seal or cover has to be removed before the cap can be accessed.

Where material is packaged in a container, the container will usually be sterilized before the material is added to it.

Where a glass container (e.g. a syringe or a vial) is used, then it can usefully be made from a borosilicate glass rather than from a soda lime glass.

### Reconstitution

Prior to administration to a patient, the invention involves reconstitution of a freeze-dried antigenic component (containing at least one meningococcal conjugate) with an aqueous component (not containing a Hib conjugate). Reconstitution can involve various steps.

If the components are present in a multi-chamber syringe then actuation of the syringe will combine the aqueous and dried materials. Where the components are present in separate containers, different mixing processes can be used. In some embodiments, aqueous material in a vial can be extracted into a syringe (e.g. via a needle), or may already be present in a syringe. The aqueous material can then be transferred from the syringe into a vial containing the lyophilised material (e.g. via a needle, which may be the same as or different from a needle previously used to extract aqueous material from a vial). The lyophilised material is thereby reconstituted and can be withdrawn (e.g. via a needle, again being the same as or different from a previously-used needle) into a syringe (e.g. the same as or different from a previously-used syringe), from which it can be injected into a patient (e.g. via a needle, again being the same as or different from a previously-used needle).

Once the lyophilised material and aqueous material have been combined and are present in a delivery device (typically a syringe) then the composition can be administered to a patient. Reconstitution will typically take place immediately prior to administration to a patient e.g. no more than 30 minutes prior to injection.

### Methods of Treatment and Administration of the Vaccine

The invention involves the co-administration of various immunogens in the form of a combination vaccine. The reconstituted compositions are suitable for administration to human patients, and the invention provides a method of raising an immune response in a patient, comprising the step of administering to the patient a composition of the invention.

The invention also provides a composition of the invention for use in medicine.

The invention also provides the use of (i) an aqueous component, comprising an immunogen, but not including a conjugate of a Haemophilus influenzae type B capsular saccharide; and (ii) a lyophilised component, comprising conjugates of capsular saccharides from meningococcal serogroups A, C, W135 and Y, in the manufacture of a medicament for administration to a patient.

The invention also provides a combination of (i) an aqueous component, comprising an immunogen, but not including a conjugate of a Haemophilus influenzae type B capsular saccharide; and (ii) a lyophilised component, comprising conjugates of capsular saccharides from meningococcal serogroups A, C, W135 and Y, for use in immunisation.

Reconstituted compositions of the invention are preferably vaccines, for use in the reduction or prevention of meningococcal meningitis and possibly further diseases e.g. tetanus, diphtheria, hepatitis B virus infection, whooping cough, pneumococcal meningitis, otitis media, etc.

Patients for receiving the compositions of the invention may be any age, but some target populations include children less than 2 years old e.g. aged between 0-12 months, patients aged between 1 and 3 months, patients who have not previously received a meningococcal conjugate vaccine, adults (i.e. 18 years and older), etc.

In order to have full efficacy, a typical primary immunization schedule for a child may involve administering more than one dose. For example, doses may be at: 0, 2 and 4 months (time 0 being the first dose); 0, 1 and 2 months; 0 and 2 months; 0, 2 and 8 months; etc. The first dose (time 0) may be administered at about 2 months of age, or sometimes (e.g. in a 0-2-8 month schedule) at around 3 months of age. Compositions can also be used as booster doses e.g. for children, in the second year of life.

Compositions of the invention can be administered by intramuscular injection e.g. into the arm, leg or buttock.

Where compositions of the invention include an aluminium-based adjuvant, settling of components may occur during storage. Aqueous compositions should therefore be shaken before and after reconstitution, prior to administration to a patient.

### Conjugation

The invention uses meningococcal conjugates in which capsular saccharides are conjugated to carrier proteins. Useful carrier proteins for covalent conjugation are bacterial toxins or toxoids, such as diphtheria toxoid or tetanus toxoid, or derivatives thereof such as the CRM197 diphtheria toxin mutant [112-114]. Other suitable carrier proteins include the N. meningitidis outer membrane protein

, synthetic peptides [116,117], heat shock proteins [118,119], pertussis proteins [120,121], cytokines [122], lymphokines [122], hormones [122], growth factors [122], artificial proteins comprising multiple human CD4⁺T cell epitopes from various pathogen-derived antigens [123] such as N19 [124], protein D from H. influenzae [125-127], pneumolysin [128] or its non-toxic derivatives [129], pneumococcal surface protein PspA [130], iron-uptake proteins [131], toxin A or B from C. difficile [132], recombinant Pseudomonas aeruginosa exoprotein A (rEPA) [133], etc.

Diphtheria toxoid (Dt), tetanus toxoid (Tt) and CRM197 are the main carriers currently in use in pediatric vaccines e.g. the Hib conjugates from GSK (e.g. as present in HIBERIX™ and INFANRIX HEXA™) use Tt as the carrier, the HIBTITER™ product uses CRM197, the pneumococcal conjugates in PREVENAR™ use CRM197, the MENJUGATE™ and MENINGITEC™ products use CRM197, and NEISVAC-C™ uses Tt.

Conjugates with a saccharide:protein ratio (w/w) of between 1:5 (i.e. excess protein) and 5:1 (i.e. excess saccharide) may be used e.g. ratios between 1:2 and 5:1 and ratios between 1:1.25 and 1:2.5.

Conjugates may be used in conjunction with free carrier protein [134], particularly where the carrier in one or more conjugate(s) is a diphtheria toxoid, tetanus toxoid or pertussis antigen.

The saccharide will typically be activated or functionalised prior to conjugation. Activation may involve, for example, cyanylating reagents such as CDAP (e.g. 1-cyano-4-dimethylamino pyridinium tetrafluoroborate [135,136, etc.]). Other suitable techniques use active esters, carbodiimides, hydrazides, norborane, p-nitrobenzoic acid, N-hydroxysuccinimide, S-NHS, EDC, TSTU; see also the introduction to reference 137). Reductive amination can be used to introduce a reactive amino group.

A process involving the introduction of amino groups into the saccharide (e.g. by replacing terminal=0 groups with -NH₂) followed by derivatisation with an adipic diester (e.g. adipic acid N-hydroxysuccinimido diester) and reaction with carrier protein can be used. In another useful reaction, a saccharide is derivatised with a cyanylating reagent, followed by coupling to a protein (direct, or after introduction of a thiol or hydrazide nucleophile group into the carrier), without the need to use a linker. Suitable cyanylating reagents include 1-cyano-4-(dimethylamino)-pyridinium tetrafluoroborate (‘CDAP’), p-nitrophenylcyanate and N-cyanotriethylammonium tetrafluoroborate (‘CTEA’).

The carrier protein may be covalently conjugated to the saccharide directly or via a linker. Various linkers are known e.g. an adipic acid linker, which may be used by coupling a free —NH₂ group (e.g. introduced to a saccharide by reductive amination) with an activated adipic acid (using, for example, diimide activation), and then coupling a protein to the resulting saccharide-adipic acid intermediate [138, 139]. Another type of linkage is a carbonyl linker, which may be formed by reaction of a free hydroxyl group of a modified glucan with CDI [140, 141] followed by reaction with a protein to form a carbamate linkage. Other linkers include β-propionamido [142], nitrophenyl-ethylamine [143], haloacyl halides [144], glycosidic linkages [145], 6-aminocaproic acid [146], N-succinimidyl-3-(2-pyridyldithio)-propionate (SPDP) [147], adipic acid dihydrazide ADH [148], C₄ to C₁₂ moieties [149], etc. Carbodiimide condensation can also be used [150]. The most preferred link between a carrier and a saccharide is via an adipic acid linker.

Saccharides will typically be covalently linked, either directly or via a linker, to a carrier via a free —NH₂ group in the carrier e.g. in a lysine side chain, an arginine side chain or at the N-terminus. Attachment via -SH is also possible e.g. in a cysteine side chain.

CRM197 conjugates of the invention may be obtained as described in reference 105.

As described in reference 151, a mixture can include one conjugate with direct saccharide/protein linkage and another conjugate with linkage via a linker. According to the invention, however, it is preferred that each conjugate includes a linker.

After conjugation, free and conjugated saccharides can be separated. There are many suitable methods for this separation, including hydrophobic chromatography, tangential ultrafiltration, diafiltration, etc. (see also refs. 152 & 153, etc.). If a vaccine comprises a given saccharide in both free and conjugated forms, the unconjugated form is usefully no more than 20% by weight of the total amount of that saccharide in the composition as a whole (e.g. ≦15%, ≦10%, ≦5%, ≦2%, ≦1%).

The amount of carrier (conjugated and unconjugated) from each conjugate may be no more than 100 μg/ml e.g. <30 μg/ml of carrier protein from each conjugate. Some compositions include a total concentration of carrier of less than 500 μg/ml e.g. <400 μg/ml, <300 μg/ml, <200 μg/ml, <100 μg/ml, <50 μg/ml, etc.

### Characteristics of Compositions of the Invention

In addition to the immunogenic components described above, compositions of the invention (both before and after mixing) will generally include non-antigenic component(s). The non-immunogenic component(s) can include carriers, adjuvants, excipients, buffers, etc., as described below.

Compositions of the invention will usually include one or more pharmaceutical carrier(s) and/or excipient(s). Sterile pyrogen-free, phosphate-buffered physiologic saline is a typical carrier. A thorough discussion of pharmaceutically acceptable excipients is available in reference 154.

To control tonicity, it is useful to include a physiological salt, such as a sodium salt. Sodium chloride (NaCl) is one such salt, which may be present at between 1 and 20 mg/ml.

Aqueous compositions (before and/or after reconstitution of lyophilised material) will generally have an osmolality of between 200 mOsm/kg and 400 mOsm/kg e.g. between 240-360 mOsm/kg, or within the range of 290-320 mOsm/kg.

Compositions of the invention may include one or more buffers. Typical buffers include: a phosphate buffer; a Tris buffer; a borate buffer; a succinate buffer; a histidine buffer; or a citrate buffer. Buffers will typically be included in the 5-20 mM range. Such buffers may be included in the aqueous and/or lyophilised components.

The pH of an aqueous composition will generally be between 5.0 and 7.5, and more typically between 5.0 and 6.0 for optimum stability, or between 6.0 and 7.0.

Compositions of the invention are preferably sterile.

Compositions of the invention are preferably non-pyrogenic e.g. containing <1 EU (endotoxin unit, a standard measure) per dose, and preferably <0.1 EU per dose.

Compositions of the invention may be gluten free.

Some vaccines of the invention are unadjuvanted. Other vaccines of the invention include adjuvant. Unadjuvanted vaccines can be made my combining unadjuvanted components. Adjuvanted vaccines can be made by combining multiple adjuvanted components, by combining adjuvanted and unadjuvanted components, or by combining unadjuvanted components with an adjuvant.

The concentration of any aluminium salts in a composition, expressed in terms of Al³⁺, is preferably less than 5 mg/ml e.g. ≦4 mg/ml, ≦3 mg/ml, ≦2 mg/ml, ≦1 mg/ml, <0.85 mg/ml, etc.

Where antigens are adsorbed, a composition may be a suspension with a cloudy appearance. This appearance means that microbial contamination is not readily visible, and so the vaccine may contain a preservative. This is particularly important when the vaccine is packaged in multidose containers. Useful preservatives for inclusion are 2-phenoxyethanol and thimerosal. It is recommended, however, not to use mercurial preservatives (e.g. thimerosal) where possible. It is preferred that compositions of the invention contain less than about 25 ng/ml mercury. Such preservatives may be included in the aqueous and/or lyophilised components. Mercury-free components and compositions are preferred, and a useful non-mercurial preservative is 2-phenoxyethanol (2-PE). 2-PE levels of less than 10 mg/ml are typical in the aqueous component e.g. between 4-7 mg/ml e.g. about 5 mg/ml, or about 6.6 mg/ml. But in some embodiments the aqueous component can be preservative-free.

Compositions of the invention may be administered to patients in 0.5 ml doses. References to 0.5 ml doses will be understood to include normal variance e.g. 0.5 ml±0.05 ml. An aqueous component used with the invention may thus have a volume of 0.5 ml.

### Adjuvants

Compositions of the invention may include an adjuvant, and this adjuvant may comprise one or more aluminium salts, and particularly an aluminium phosphate adjuvant and/or an aluminium hydroxide adjuvant. Antigenic components used to prepare compositions of the invention may include aluminium adjuvants before being used i.e. they are ‘pre-mixed’ or ‘pre-adsorbed’ to the adjuvant(s). The adjuvant will usually be present in the aqueous component but may also (or alternatively) be present in the lyophilised component.

Aluminium adjuvants currently in use are typically referred to either as “aluminium hydroxide” or as “aluminium phosphate” adjuvants. These are names of convenience, however, as neither is a precise description of the actual chemical compound which is present (e.g. see chapter 9 of reference 155). The invention can use any of the “hydroxide” or “phosphate” salts that are in general use as adjuvants.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-3100 cm⁻¹ (chapter 9 of ref. 155).

The adjuvants known as “aluminium phosphate” are typically aluminium hydroxyphosphates, often also containing a small amount of sulfate. They may be obtained by precipitation, and the reaction conditions and concentrations during precipitation can influence the degree of substitution of phosphate for hydroxyl in the salt. Hydroxyphosphates generally have a PO₄/Al molar ratio between 0.3 and 0.99. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls (chapter 9 of ref. 155).

The Pa₄/Al³⁺ molar ratio of an aluminium phosphate adjuvant will generally be between 0.3 and 1.2, preferably between 0.8 and .1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate. Typical diameters of the particles are in the range 0.5-20 μm (e.g. about 5-10 μm) after any antigen adsorption.

The PZC of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

An aluminium phosphate solution used to prepare a composition of the invention may contain a buffer (e.g. a phosphate or a histidine or a Tris buffer), but this is not always necessary. The aluminium phosphate solution is preferably sterile and pyrogen-free. The aluminium phosphate solution may include free aqueous phosphate ions e.g. present at a concentration between 1.0 and 20 mM, preferably between 5 and 15 mM, and more preferably about 10 mM. The aluminium phosphate solution may also comprise sodium chloride. The concentration of sodium chloride is preferably in the range of 0.1 to 100 mg/ml (e.g. 0.5-50 mg/ml, 1-20 mg/ml, 2-10 mg/ml) and is more preferably about 3+1 mg/ml. The presence of NaCl facilitates the correct measurement of pH prior to adsorption of antigens.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Concentrations of conjugates are defined herein in terms of mass of saccharide, in order to avoid variation due to choice of carrier.

Where an antigen is described as being “adsorbed” to an adjuvant, it is preferred that at least 50% (by weight) of that antigen is adsorbed e.g. 50%, 60%, 70%, 80%, 90%, 95%, 98% or more. It is preferred that diphtheria toxoid and tetanus toxoid are both totally adsorbed i.e. none is detectable in supernatant. Total adsorption of HBsAg is also preferred.

Where animal (and particularly bovine) materials are used in the culture of cells, they should be obtained from sources that are free from transmissible spongiform encaphalopathies (TSEs), and in particular free from bovine spongiform encephalopathy (BSE).

### MODES FOR CARRYING OUT THE INVENTION

Capsular saccharides are purified from meningococci from serogroups A, C, W135 and Y following the procedures disclosed in references 91 and 105. They are conjugated to CRM197 following the procedures disclosed in references 91 and 105. In alternative embodiments they are conjugated to tetanus toxoid.

The conjugates are mixed and then lyophilised to give final amounts per dose of 12 μg MenA and 6 μg of each of MenC, MenW135 and MenY. Sucrose is included at 30 mg/dose for stabilisation.

The total and free saccharide contents of each of the CRM-MenA, CRM-MenC, CRM-MenY and CRM-MenW conjugates were confirmed using high performance anion exchange chromatography coupled with pulsed amperometric detection (HPAEC-PAD) and by colorimetric methods. Molecular size distribution was determined using size exclusion chromatography coupled to PAD and capillary zone electrophoresis (CZE), to monitor the integrity of these conjugates after lyophilisation. The results indicated that lyophilisation did not have any negative impact on saccharide content or molecular size distribution of the glycoconjugates when compared to pre-lyophilised conjugates.

NMR was also used to analyse the identity and stability conjugates, both on monovalent bulks and also in the final combined mixture (after reconstitution into aqueous form). Since each lyophilized combination contains a large excess of sucrose, samples were dialysed at 4° C. for 48 hours with four changes of 10 mM sodium phosphate buffer, pH 7.2 to remove the sucrose.

An identity test was developed by selecting a 0.7 ppm restricted window (from the down-field value at 5.6 ppm to the up-field value at 4.9 ppm) where the proton anomeric signals of the meningococcal conjugates were detected and assigned. Selecting a restricted spectral region, the assay was very simple but could identify all the conjugated polysaccharide antigens in the combined vaccine, detecting two signals for MenA and one signal for each of MenC, MenW 135 and MenY.

The combined 4-valent conjugate lyophilisate is reconstituted with an aqueous vaccine such as INFANRIX PENTA™, DAPTACEL™ or PEDIACEL™.

It will be understood that the invention will be described by way of example only, and that modifications may be made whilst remaining within the scope and spirit of the invention.

### REFERENCES

[1] Vaccines. (eds. Plotkin & Orenstein). 4th edition, 2004, ISBN: 0-7216-9688-0.
[2] WO02/00249.
[3] WO2005/032583.
[4] WO2008/149238.
[5] Sesardic et al. (2001) Biologicals 29:107-22.
[6] NIBSC code: 98/560.
[7] Module 1 of WHO's The immunological basis for immunization series (Galazka).
[8] NIBSC code: 69/017.
[9] NIBSC code: DIFT.
[10] Sesardic et al. (2002) Biologicals 30:49-68.
[11] NIBSC code: 98/552.
[12] NIBSC code: TEFT.
[13] Rappuoli et al. (1991) TIBTECH 9:232-238.
[14] Nencioni et al. (1991) Infect Immun. 59(2): 625-30.
[15] Vanlandschoot et al. (2005) J Gen Virol 86:323-31.
[16] WO93/24148.
[17] WO2007/007344.
[18] WO2008/044611.
[19] Module 6 of WHO's The immunological basis for immunization series (Robertson)
[20] WO2008/028957.
[21] WHO Technical Report Series No. 927, 2005. Pages 64-98.
[22] US-2008/0102498.
[23] US-2006/0228381.
[24] US-2007/0231340.
[25] US-2007/0184072.
[26] US-2006/0228380.
[27] Research Disclosure, 453077 (Jan 2002)
[28] EP-A-0378881.
[29] EP-A-0427347.
[30] WO93/17712
[31] WO94/03208.
[32] WO98/58668.
[33] EP-A-0471177.
[34] WO91/01146
[35] Falugi et al. (2001) Eur J Immunol 31:3816-3824.
[36] Baraldo et al. (2004) Infect Immun 72(8):4884-7.
[37] EP-A-0594610.
[38] Ruan et al. (1990) J Immunol 145:3379-3384.
[39] WO00/56360.
[40] Kuo et al. (1995) Infect Immun 63:2706-13.
[41] Michon et al. (1998) Vaccine. 16:1732-41.
[42] WO02/091998.
[43] WO01/72337
[44] WO00/61761.
[45] WO00/33882
[46] EP-A-0372501.
[47] WO2007/071707
[48] Bethell G. S. et al., J. Biol. Chem., 1979, 254, 2572-4
[49] Hearn M. T. W., J. Chromatogr., 1981, 218, 509-18
[50] WO2007/000343.
[51] Mol. Immunol., 1985, 22, 907-919
[52] EP-A-0208375
[53] WO00/10599
[54] Gever et al., Med. Microbiol. Immunol, 165 : 171-288 (1979).
[55] U.S. Pat. No. 4,057,685.
[56] U.S. Pat. Nos. 4,673,574; 4,761,283; 4,808,700.
[57] U.S. Pat. No. 4,459,286.
[58] U.S. Pat. No. 5,204,098
[59] U.S. Pat. No. 4,965,338
[60] U.S. Pat. No. 4,663,160.
[61] US-2007/0184071.
[62] Jodar et al. (2003) Vaccine 21:3265-72.
[63] WO2008/135514.
[64] WO02/09643.
[65] Katial et al. (2002) Infect. Immun. 70:702-707.
[66] U.S. Pat. No. 6,180,111.
[67] WO01/34642.
[68] WO2004/019977.
[69] European patent 0011243.
[70] Fredriksen et al. (1991) NIPH Ann. 14(2):67-80.
[71] WO01/91788.
[72] WO2005/004908.
[73] Maiden et al. (1998) PNAS USA 95:3140-3145.
[74] WO98/56901.
[75] WO2006/081259.
[76] Claassen et al. (1996) 14(10):1001-8.
[77] WO01/52885.
[78] WO00/25811.
[79] WO01/09350.
[80] WO02/09746.
[81] WO02/062378.
[82] WO2004/014417.
[83] WO2007/144316.
[84] WO2007/144317.
[85] van den Dobbelsteen et al. (2007) Vaccine 25:2491-6.
[86] W. H. O. Tech. Rep. Ser. 594:51, 1976.
[87] Jones (2001) Curr Opin Investig Drugs 2:47-49.
[88] Costantino et al. (1992) Vaccine 10:691-8.
[89] Lieberman et al. (1996) JAMA 275:1499-503.
[90] WO02/058737.
[91] WO03/007985.
[92] Rennels et al. (2002) Pediatr Infect Dis J 21:978-979.
[93] Campbell et al. (2002) J Infect Dis 186:1848-1851.
[94] WO03/080678.
[95] Glode et al. (1979) J Infect Dis 139:52-56
[96] WO94/05325; U.S. Pat. No. 5,425,946.
[97] Arakere & Frasch (1991) Infect. Immun. 59:4349-4356.
[98] Michon et al. (2000) Dev. Biol. 103:151-160.
[99] Rubinstein & Stein (1998) J. Immunol. 141:4357-4362.
[100] WO2005/033148.
[101] WO2007/000314.
[102] WO2007/000341.
[103] WO2008/011201.
[104] WO2004/067030.
[105] Bardotti et al. (2008) Vaccine 26:2284-96.
[106] WO2007/000327.
[107] WO2007/000322.
[108] Bröker et al. (2009) Vaccine 27:5574-80.
[109] Knuf et al. (2009) Vaccine doi:10.1016/j.vaccine.2009.10.064.
[110] Pozsgay (2008) Curr Top Med Chem 8 :126-140.
[111] Berkin et al. (2002) Chemistry 8:4424-33.
[112] Anonymous (Jan 2002) Research Disclosure, 453077.
[113] Anderson (1983) Infect Immun 39(1):233-238.
[114] Anderson et al. (1985) J Clin Invest 76(1):52-59.
[115] EP-A-0372501.
[116] EP-A-0378881.
[117] EP-A-0427347.
[118] WO93/17712
[119] WO94/03208.
[110] WO98/58668.
[121] EP-A-0471177.
[122] WO91/01146
[123] Falugi et al. (2001) Eur J Immunol 31:3816-3824.
[124] Baraldo et al. (2004) Infect Immun 72(8):4884-7.
[125] EP-A-0594610.
[126] Ruan et al. (1990) J Immunol 145:3379-3384.
[127] W000/56360.
[128] Kuo et al. (1995) Infect Immun 63:2706-13.
[129] Michon et al. (1998) Vaccine. 16:1732-41.
[130] WO02/091998.
[131] WO01/72337
[132] WO00/61761.
[133] WO00/33882
[134] WO96/40242
[135] Lees et al. (1996) Vaccine 14:190-198.
[136] WO95/08348.
[137] WO2006/075170.
[138] Mol. Immunol., 1985, 22, 907-919
[139] EP-A-0208375
[140] Bethell G. S. et al., J. Biol. Chem., 1979, 254, 2572-4
[141] Hearn M. T. W., J. Chromatogr., 1981, 218, 509-18
[142] WO00/10599
[143] Geyer et al., Med. Microbiol. Immunol, 165: 171-288 (1979).
[144] U.S. Pat. No. 4,057,685.
[145] U.S. Pat. Nos. 4,673,574; 4,761,283; 4,808,700.
[146] U.S. Pat. No. 4,459,286.
[147] U.S. Pat. No. 5,204,098
[148] U.S. Pat. No. 4,965,338
[149] U.S. Pat. No. 4,663,160.
[150] WO2007/000343.
[151] WO2007/000342.
[152] Lei et al. (2000) Dev Biol (Basel) 103:259-264.
[153] WO00/38711; U.S. Pat. No. 6,146,902.
[154] Gennaro (2000) Remington: The Science and Practice of Pharmacy. 20th edition, ISBN: 0683306472.
[155] Vaccine Design: The Subunit and Adjuvant Approach (eds. Powell & Newman) Plenum Press 1995 (ISBN 0-306-44867-X).

1. A kit comprising: (i) an aqueous component, comprising an immunogen, but not including a conjugate of a Haemophilus influenzae type B capsular saccharide; and (ii) a lyophilised component, comprising conjugates of capsular saccharides from meningococcal serogroups A, C, W135 and Y.

2. A method for preparing a combined vaccine, comprising the step of combining (i) an aqueous component, comprising an immunogen, but not including a conjugate of a Haemophilus influenzae type B capsular saccharide; and (ii) a lyophilised component, comprising conjugates of capsular saccharides from meningococcal serogroups A, C, W135 and Y.

3. The kit of claim 1, wherein the aqueous component includes a diphtheria toxoid.

4. The kit of claim 1, wherein the aqueous component includes a tetanus toxoid.

5. The kit of claim 1, wherein the aqueous component includes a cellular B. pertussis antigen.

6. The kit of claim 1, wherein the aqueous component includes at least one acellular B. pertussis antigen.

7. The kit of claim 1, wherein the aqueous component includes hepatitis B virus surface antigen (‘HBsAg’).

8. The kit of claim 1, wherein the aqueous component includes an inactivated poliovirus vaccine (‘IPV’).

9. The kit of claim 1, wherein the aqueous component includes conjugated capsular saccharide(s) from at least one serotype of Streptococcus pneumoniae.

10. The kit of claim 1, wherein the aqueous component includes vesicles from a serogroup B meningococcus.

11. The kit of claim 1, wherein the aqueous component includes a hepatitis A virus antigen (‘HAV’).

12. The kit of claim 1, wherein the aqueous component includes a human papillomavirus antigen.

13. The kit of claim 1, wherein the aqueous component includes a diphtheria toxoid and a tetanus toxoid.

14. The kit of claim 1, wherein the aqueous component includes a diphtheria toxoid, a tetanus toxoid and a cellular B. pertussis antigen.

15. The kit of claim 1, wherein the aqueous component includes a diphtheria toxoid, a tetanus toxoid and an acellular B. pertussis antigen.

16. The kit of laim 1, wherein the aqueous component includes a diphtheria toxoid, a tetanus toxoid, a cellular B. pertussis antigen, and HBsAg.

17. The kit of claim 1, wherein the aqueous component includes a diphtheria toxoid, a tetanus toxoid, an acellular B. pertussis antigen, and HBsAg.

18. The kit of claim 1, wherein the aqueous component includes a diphtheria toxoid, a tetanus toxoid, a cellular B. pertussis antigen, and IPV.

19. The kit of claim 1, wherein the aqueous component includes a diphtheria toxoid, a tetanus toxoid, an acellular B. pertussis antigen, and IPV.

20. The kit of claim 1, wherein the aqueous component includes a diphtheria toxoid, a tetanus toxoid, a cellular B. pertussis antigen, HBsAg and IPV.

21. The kit of claim 1, wherein the aqueous component includes a diphtheria toxoid, a tetanus toxoid, an acellular B. pertussis antigen, HBsAg and IPV.

22. The kit of claim 1, wherein the aqueous component includes vesicles from a serogroup B meningococcus and conjugated capsular saccharide(s) from at least one serotype of Streptococcus pneumoniae.

23. The kit of claim 1, wherein the aqueous component includes a HAV antigen and HBsAg.

24. The kit of claim 1, wherein the mass ratio of saccharides from serogroups A, C, W135 andY is 1:1:1:1, 2:1:1:1, 1:4:1:1, 1:2:1:1 or 2:2:1:1 (A:C:W135:Y).

25. The kit of claim 1, wherein the aqueous component includes an adjuvant.

26. The kit of claim 25, wherein the adjuvant comprises aluminium hydroxide and/or aluminium phosphate.

27. The kit of claim 1, wherein the lyophilised component includes an adjuvant.

28. The kit of claim 27, wherein the adjuvant comprises aluminium hydroxide and/or aluminium phosphate.

29. The kit of claim 1, wherein the lyophilised component is adjuvant-free.

30. A combined vaccine comprising: (i) conjugates of capsular saccharides from meningococcal serogroups A, C, W135 and Y; (ii) at least one further immunogen; and (iii) at least one lyophilisation stabiliser, provided that the vaccine does not include a conjugate of a Haemophilus influenzae type B capsular saccharide,

31. A method of raising an immune response in a patient, comprising the step of administering to the patient the combined vaccine of claim 30.
